(19) United States
(12) Patent Application Publication
(10) Pub. No.: US 2025/0152244 A1
(43) Pub. Date: May 15, 2025

(54) AUTOMATED FRAME FEATURE DETECTION IN FRAME MATCHING PROCESS USING MACHINE LEARNING

(71) Applicant: DEPUY SYNTHES PRODUCTS, INC., Raynham, MA (US)

(72) Inventor: Bernd GUTMANN, Hassloch (DE)

(21) Appl. No.: 18/928,316

(22) Filed: Oct. 28, 2024

Related U.S. Application Data
(60) Provisional application No. 63/597,558, filed on Nov. 9, 2023.

(51) Int. Cl. A61B 34/10; A61B 6/50; G06T 3/40; G06T 3/60; G06T 5/70; G06V 10/44; G16H 30/40

(52) U.S. Cl. CPC A61B 34/10; A61B 6/505; G06T 3/40; G06T 3/60; G06T 5/70; G06V 10/44; G16H 30/40; A61B 2034/102; A61B 2034/105; A61B 2034/108; G06T 2207/20081; G06T 2207/20084; G06V 2201/07

(57) ABSTRACT

An automatic feature matching system for orthopedic fixators includes a frame generator, an artificial X-ray generator, a neural network trainer using neural network training data. The frame generator is configured to generate a simulation of an orthopedic fixator system. The artificial X-ray generator is configured to generate a plurality of artificial X-ray images including the generated simulation of the orthopedic fixator system in the plurality of artificial X-ray image, wherein the plurality of artificial X-ray images are labelled. The neural network training data includes the plurality of labelled artificial X-ray images. The neural network trainer is configured to be trained based on the plurality of labelled artificial X-ray images. The neural network trainer is configured to generate a frame detection neural network that is configured to detect orthopedic fixator system features in real X-ray images input into the frame detection neural network.

### FIELD OF THE DISCLOSURE

Various exemplary embodiments disclosed herein relate to automated frame feature detection in frame matching process using machine learning such as neural networks.

### BACKGROUND

Techniques used to treat fractures and/or deformities of anatomical structures, such as bones, can include the use of external fixators, such as hexapods and other fixation frames, which are surgically mounted to anatomical structure segments on opposed sides of a fracture site. A pair of radiographic images is taken of the fixator and anatomical structure segments at the fracture site. Data from the images is then manipulated to construct a three-dimensional representation of the fixator and the anatomical structures segments that can be used in developing a treatment plan, which may for example comprise realigning the anatomical structure segments through adjustments to the fixator.

Existing techniques for controlling fixator manipulation may, however, involve a number of limitations that may introduce inefficiency, complication, and unreliability. For example, some conventional techniques may rely on a surgeon or other user to indicate locations of certain fixator elements, such as hinges, within images that are displayed in a graphical user interface of a computer. However, it may often be difficult for the user to identify and mark positions of the hinges and other fixator elements within the images. In particular, depending upon the location and orientation from which an image is captured, hinges and other fixator elements may not be identified easily, such as because they may wholly or partially overlap one another or may otherwise be obscured within the images. This may make it cumbersome for the user to identify the fixator elements, thereby increasing time required to identify the elements, increasing the probability of errors, and reducing the reliability of the calculations. This may reduce the reliability of the treatment plan, possibly resulting in improper alignment of anatomical structures segments during the healing process, compromised union between the anatomical structure segments, necessitating additional rounds of radiographic imaging to facilitate alignment corrections, or even necessitating additional surgical procedures.

### SUMMARY

A summary of various exemplary embodiments is presented below.

Various embodiments of the present invention relate to an automatic feature matching system for orthopedic fixators, including: a frame generator configured to generate a model (i.e., a simulation of the physical object, not to be confused with a deep learning model) of an orthopedic fixator system; an artificial X-ray generator configured to generate a plurality of artificial X-ray images including the model (i.e., the simulation) of generated orthopedic fixator system in the plurality of artificial X-ray image, wherein the plurality of artificial X-ray images are labelled; neural network training data including the plurality of artificial X-ray images; and a neural network trainer configured to be trained based on the plurality of artificial X-ray images; wherein the neural network trainer is configured to generate a frame detection neural network that is configured to detect orthopedic fixator system features in real X-ray images input into the frame detection neural network.

Various embodiments of the present invention provide an automatic feature matching system for orthopedic fixators. The system includes a frame generator configured to generate a simulation of an orthopedic fixator system. The system also includes an artificial X-ray generator configured to generate a plurality of artificial X-ray images including the generated simulation of the orthopedic fixator system in the plurality of artificial X-ray image, wherein the plurality of artificial X-ray images are labelled. The system also includes a neural network training data including the plurality of labelled artificial X-ray images. The system also includes a neural network trainer configured to be trained based on the plurality of labelled artificial X-ray images. The neural network trainer is configured to generate a frame detection neural network that is configured to detect orthopedic fixator system features in real X-ray images input into the frame detection neural network.

According to embodiments of the present invention the frame generator randomly selects orthopedic fixator parameters and configurations based upon clinical data.

According to embodiments of the present invention the frame generator further generates anatomy elements connected to the generated orthopedic fixator.

According to embodiments of the present invention the artificial X-ray generator generates multiple X-rays of the generated orthopedic frame using different aspects and X-ray parameters.

According to embodiments of the present invention, labelled clinical images and datasets of orthopedic fixators are also provided. Labeled clinical images can be mixed with dataset or can be used for transfer learning of a model, trained by the artificial X-Rays,

According to embodiments of the present invention the neural network training data includes the labelled clinical images of orthopedic fixators.

According to embodiments of the present invention the at least a subset of the labelled clinical images of orthopedic fixators are used by the neural network trainer to validate the frame detection neural network.

According to embodiments of the present invention the at least a subset of the labelled clinical images of orthopedic fixators are used by the neural network trainer to test the frame detection neural network.

According to embodiments of the present invention the at least a subset of the labelled clinical images of orthopedic fixators are used by the neural network trainer to validate the frame detection neural network.

According to embodiments of the present invention the generated frame detection neural network is configured to detect hinges and their positions in the images input into the frame detection neural network.

According to embodiments of the present invention the generated frame detection neural network is configured to adjustment members in the images input into the frame detection neural network.

According to embodiments of the present invention the generated frame detection neural network is configured to detect hinges and adjustment members in the images input into the frame detection neural network.

According to embodiments of the present invention the generated frame detection neural network is configured to detect a full frame of the orthopedic fixator in the images input into the frame detection neural network.

According to embodiments of the present invention the generated frame detection neural network is configured to detect features in two images input into the frame detection neural network.

According to embodiments of the present invention an image processor is configured to apply image processing to one or more of said plurality of labelled artificial X-ray images to generate a plurality of processed labelled artificial X-ray images. The neural network training data further includes the plurality of processed labelled artificial X-ray images, and the neural network trainer is configured to be trained based on the plurality of labelled artificial X-ray images and/or the plurality of processed labelled artificial X-ray images.

According to embodiments of the present invention the image processor is configured to apply image processing including at least one of blurring, aspect ratio, noise, annotations, brightness, contrast, rotation, scaling, translation, color, and cropping.

According to embodiments of the present invention the neural network trainer generates one or more trained models configured to detect orthopedic fixators in an image.

According to embodiments of the present invention the trained model is used to train a new model for detecting orthopedic fixators using a new neural network training dataset.

According to embodiments of the present invention, a method of generating a deep learning model for automatic feature matching of orthopedic fixators includes steps of generating a simulation of an orthopedic fixator system; generating a plurality of artificial X-ray images including the generated simulation of the orthopedic fixator system in the plurality of artificial X-ray image, wherein the plurality of artificial X-ray images are labelled; creating neural network training data including the plurality of labelled artificial X-ray images; and training a neural network model based on the plurality of labelled artificial X-ray images to generate a frame detection neural model that is configured to detect orthopedic fixator system features in real X-ray images input into the frame detection neural network.

According to embodiments of the present invention the generating a simulation step randomly selects orthopedic fixator parameters and configurations based upon clinical data.

According to embodiments of the present invention the generating a simulation step further generates anatomy elements connected to the generated orthopedic fixator.

According to embodiments of the present invention the generating a plurality of artificial X-ray images step generates multiple X-rays of the generated orthopedic frame using different aspects and X-ray parameters.

According to embodiments of the present invention labelled clinical images and datasets of orthopedic fixators are provided.

According to embodiments of the present invention the neural network training data includes the labelled clinical images of orthopedic fixators.

According to embodiments of the present invention the at least a subset of the labelled clinical images of orthopedic fixators are used by the neural network trainer to validate the frame detection neural network.

According to embodiments of the present invention the at least a subset of the labelled clinical images of orthopedic fixators are used by the neural network trainer to test the frame detection neural network.

According to embodiments of the present invention the generated frame detection neural model is configured to detect hinges and their positions in the images input into the frame detection neural network.

According to embodiments of the present invention the generated frame detection neural model is configured to adjustment members in the images input into the frame detection neural network.

According to embodiments of the present invention the generated frame detection neural model is configured to detect hinges and adjustment members in the images input into the frame detection neural network.

According to embodiments of the present invention the generated frame detection neural model is configured to detect a full frame of the orthopedic fixator in the images input into the frame detection neural network.

According to embodiments of the present invention the generated frame detection neural model is configured to detect features in two images input into the frame detection neural network.

According to embodiments of the present invention the image processing includes one or more of said plurality of labelled artificial X-ray images to generate a plurality of processed labelled artificial X-ray images. The neural network training data further includes the plurality of processed labelled artificial X-ray images, and the model is based on the plurality of labelled artificial X-ray images and/or the plurality of processed labelled artificial X-ray images.

According to embodiments of the present invention the image processing includes at least one of blurring, aspect ratio, noise, annotations, brightness, contrast, rotation, scaling, translation, color, and cropping.

According to embodiments of the present invention the method generates one or more trained models configured to detect orthopedic fixators in an image.

According to embodiments of the present invention the trained model is used to train a new model for detecting orthopedic fixators using a new neural network training dataset.

The foregoing has outlined rather broadly the features and technical advantages of examples according to the disclosure in order that the detailed description that follows may be better understood. Additional features and advantages will be described hereinafter. The conception and specific examples disclosed may be readily utilized as a basis for modifying or designing other structures for carrying out the same purposes of the present disclosure. Such equivalent constructions do not depart from the scope of the appended claims. Characteristics of the concepts disclosed herein, both their organization and method of operation, together with associated advantages will be better understood from the following description when considered in connection with the accompanying figures. Each of the figures is provided for the purposes of illustration and description, and not as a definition of the limits of the claims.

### BRIEF DESCRIPTION OF DRAWINGS

So that the above-recited features of the present disclosure can be understood in detail, a more particular description, briefly summarized above, may be had by reference to aspects, some of which are illustrated in the appended drawings. It is to be noted, however, that the appended drawings illustrate only certain typical aspects of this disclosure and are therefore not to be considered limiting of its scope, for the description may admit to other equally effective aspects. The same reference numbers in different drawings may identify the same or similar elements.

FIG. 1 is a perspective view of a fixation assembly positioned for imaging in accordance with an embodiment.

FIG. 2 is a perspective view of an example imaging process of the fixation assembly illustrated in FIG. 1.

FIGS. 3A and 3B illustrate generally anterior and lateral view X-rays with user annotations on the adjustable length struts.

FIG. 4A illustrates the same lateral view as FIG. 3B with overlapping universal joints highlighted.

FIGS. 4B and 4C illustrate the specific regions of the image in FIG. 4A that illustrate where universal joints overlap and are difficult to identify and annotate.

FIG. 5 illustrates an automatic feature matching system and method using neural networks.

FIG. 6 illustrates a block diagram of an artificial X-ray generator.

FIG. 7 illustrates an artificial X-ray system.

FIG. 8 illustrates a X-ray path passing through different objects on its way to the artificial X-ray detector.

FIG. 9 illustrates an exemplary hardware diagram for implementing automatic feature matching system, frame generator, artificial X-ray generator, neural network trainer, full frame detection neural network, adjustment member detection neural network, image detection neural network, or artificial X-ray system.

### DETAILED DESCRIPTION

Various aspects of the disclosure are described more fully hereinafter with reference to the accompanying drawings. This disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the disclosure disclosed herein, whether implemented independently of or combined with any other aspect of the disclosure. For example, an apparatus may be implemented or a method may be practiced using any number of the aspects set forth herein. In addition, the scope of the disclosure is intended to cover such an apparatus or method which is practiced using other structure, functionality, or structure and functionality in addition to or other than the various aspects of the disclosure set forth herein. It should be understood that any aspect of the disclosure disclosed herein may be embodied by one or more elements of a claim.

Several aspects of hinge detection and artificial X-ray generation systems will now be presented with reference to various apparatuses and techniques. These apparatuses and techniques will be described in the following detailed description and illustrated in the accompanying drawings by various blocks, modules, components, circuits, steps, processes, algorithms, and/or the like (collectively referred to as “elements”). These elements may be implemented using hardware, software, or combinations thereof. Whether such elements are implemented as hardware or software depends upon the particular application and design constraints imposed on the overall system.

For convenience, the same or equivalent elements in the various embodiments illustrated in the drawings have been identified with the same reference numerals. Certain terminology is used in the following description for convenience only and is not limiting. The words “right”, “left”, “top” and “bottom” designate directions in the drawings to which reference is made. The words “inward”, “inwardly”, “outward”, and “outwardly” refer to directions toward and away from, respectively, the geometric center of the device and designated parts thereof. The terminology intended to be non-limiting includes the above-listed words, derivatives thereof and words of similar import.

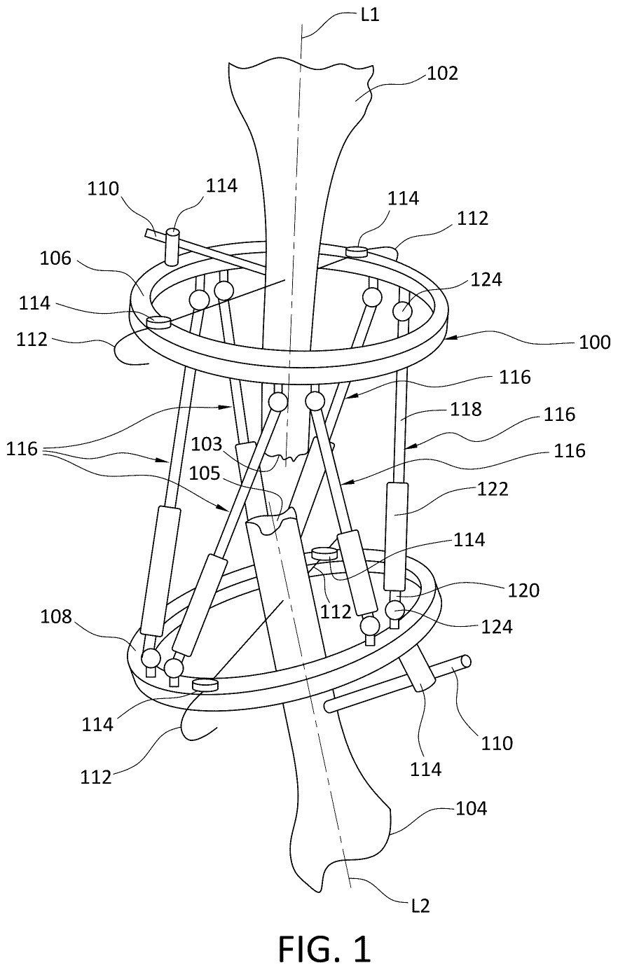

FIG. 1 is a perspective view of a fixation assembly positioned for imaging in accordance with an embodiment. Referring initially to FIG. 1, bodily tissues, for instance first and second anatomical structure segments 102, 104, can be aligned and/or oriented to promote union or other healing between the bodily tissues. Anatomical structures may include, for example, anatomical tissue and artificial anatomical implants. Anatomical tissue may include, for example, bone or other tissue in the body. The alignment and/or orientation of the bodily tissues can be achieved by connecting the bodily tissues to an adjustable fixation apparatus, such as orthopedic fixator 100. The orthopedic fixator can comprise an external fixation apparatus that includes a plurality of discrete fixator members that remain external to the patient's body, but that are attached to respective discreet bodily tissues, for example with minimally invasive attachment members. A fixation apparatus may include, for example, a distraction osteogenesis ring system, a hexapod, or a Taylor spatial frame. By adjusting the spatial positioning of the fixator members with respect to each other, the respective bodily tissues attached thereto can be reoriented and/or otherwise brought into alignment with each other, for example to promote union between the bodily tissues during the healing process. The use of external orthopedic fixators in combination with the imagery analysis and positioning techniques described herein can be advantageous in applications where direct measurement and manipulation of the bodily tissues is not possible, where limited or minimally invasive access to the bodily tissues is desired, or the like. Some examples of orthopedic fixators and their use for correcting deformities of anatomical structure segments, as well as techniques for performing imagery analysis on the fixators and anatomical structure segments are described in U.S. Pat. No. 9,642,649, entitled “ORTHOPEDIC FIXATION WITH IMAGERY ANALYSIS,” issued on May 9, 2017, the contents of which is hereby incorporated by reference for all purposes as if fully set forth herein.

The fixator members can be connected to each other via adjustment members with the adjustment members configured to facilitate the spatial repositioning of the fixator members with respect to each other. For example, in the illustrated embodiment, the orthopedic fixator 100 includes a pair of fixator members in the form of an upper fixator ring 106 and a lower fixator ring 108. The fixator rings 106, 108 can be constructed the same or differently. For instance, the fixator rings 106, 108 can have diameters that are the same or different. Similarly, the fixator rings 106, 108 can be constructed with varying cross sectional diameters, thicknesses, etc. It should be appreciated that the fixator members of the orthopedic fixator 100 are not limited to the illustrated upper and lower fixator rings 106, 108, and that the orthopedic fixator 100 can be alternatively constructed. For example, additional fixator rings can be provided and interconnected with the fixator ring 106 and/or 108. It should further be appreciated that the geometries of the fixator members are not limited to rings, and that at least one, such as all of the fixator members can be alternatively constructed using any other suitable geometry.

The first and second anatomical structure segments 102, 104 can be rigidly attached to the upper and lower fixator rings 106, 108, respectively, with attachment members that can be mounted to the fixator rings 106, 108. For example, in the illustrated embodiment, attachment members are provided in the form of attachment rods 110 and attachment wires 112.

The rods 110 and the wires 112 extend between proximal ends attached to mounting members 114 that are mounted to the fixator rings 106, 108, and opposed distal ends that are inserted into or otherwise secured to the anatomical structure segments 102, 104. The mounting members 114 can be removably mounted to the fixator rings 106, 108 at predefined points along the peripheries of the fixator rings 106, 108, for example by disposing them into threaded apertures defined by the fixator rings. With respect to each fixator ring 106, 108, the mounting members 114 can be mounted to the upper surface of the ring, the lower surface of the ring, or any combination thereof. It should be appreciated that the attachment members are not limited to the configuration of the illustrated embodiment. For example, any number of attachment members, such as the illustrated rods 110 and wires 112 and any others, can be used to secure the anatomical structure segments to respective fixator members as desired. It should further be appreciated that one or more of the attachment members, for instance the rods 110 and/or wires 112, can be alternatively configured to mount directly to the fixator rings 106, 108, without utilizing mounting members 114.

The upper and lower fixator rings 106, 108 can be connected to each other by one or more adjustment members. Any number of the adjustment members can be configured to allow the spatial positioning of the fixator rings with respect to each other to be adjusted. For example, in the illustrated embodiment, the upper and lower fixator rings 106, 108 are connected to each other with a plurality of adjustment members provided in the form of adjustable length struts 116. It should be appreciated that the construction of the orthopedic fixator 100 is not limited to the six struts 116 of the illustrated embodiment, and that more or fewer struts can be used as desired.

Each of the adjustable length struts 116 can comprise opposed upper and lower strut arms 118, 120. Each of the upper and lower strut arms 118, 120 have proximal ends disposed in a coupling member, or sleeve 122, and opposed distal ends that are coupled to universal joints 124 mounted to the upper and lower fixator rings 106, 108, respectively. The universal joints of the illustrated embodiment are disposed in pairs spaced evenly around the peripheries of the upper and lower fixator rings 106, 108, but can be alternatively placed in any other locations on the fixator rings as desired. The universal joints 124 may also be called hinges.

The proximal ends of the upper and lower strut arms 118, 120 of each strut 116 can have threads defined thereon that are configured to be received by complementary threads defined in the sleeve 122, such that when the proximal ends of the upper and lower strut arms 118, 120 of a strut 116 are received in a respective sleeve 122, rotation of the sleeve 122 causes the upper and lower strut arms 118, 120 to translate within the sleeve 122, thus causing the strut 116 to be elongated or shortened, depending on the direction of rotation. Thus, the length of each strut 116 can be independently adjusted with respect to the remaining struts. It should be appreciated that the adjustment members are not limited to the length adjustable struts 116 of the illustrated embodiment, and that the adjustment members can be alternatively constructed as desired, for example using one or more alternative geometries, alternative length adjustment mechanisms, and the like.

The adjustable length struts 116 and the universal joints 124 by which they are mounted to the upper and lower fixator rings 106, 108, allow the orthopedic fixator 100 to function much like a Stewart platform, and more specifically like a distraction osteogenesis ring system, a hexapod, or a Taylor spatial frame. That is, by making length adjustments to the struts 116, the spatial positioning of the upper and lower fixator rings 106, 108, and thus the anatomical structure segments 102, 104 can be altered. For example, in the illustrated embodiment the first anatomical structure segment 102 is attached to the upper fixator ring 106 and the second anatomical structure segment 104 is attached to the lower fixator ring 108. It should be appreciated that attachment of the first and second anatomical structure segments 102, 104 to the upper and lower fixator rings 106, 108 is not limited to the illustrated embodiment (e.g., where the central longitudinal axes L1, L2 of the first and second anatomical structure segments 102, 104 are substantially perpendicular to the respective planes of the upper and lower fixator rings 106, 108), and that a surgeon has complete flexibility in aligning the first and second anatomical structure segments 102, 104 within the upper and lower fixator rings 106, 108 when configuring the orthopedic fixator 100.

By varying the length of one or more of the struts 116, the upper and lower fixator rings 106, 108, and thus the anatomical structure segments 102 and 104 can be repositioned with respect to each other such that their respective longitudinal axes L1, L2 are substantially aligned with each other, and such that their respective fractured ends 103, 105 abut each other, so as to promote union during the healing process. It should be appreciated that adjustment of the struts 116 is not limited to the length adjustments as described herein, and that the struts 116 can be differently adjusted as desired. It should further be appreciated that adjusting the positions of the fixator members is not limited to adjusting the lengths of the length adjustable struts 116, and that the positioning of the fixator members with respect to each other can be alternatively adjusted, for example in accordance the type and/or number of adjustment members connected to the fixation apparatus.

Repositioning of the fixator members of an orthopedic fixation apparatus, such as orthopedic fixator 100, can be used to correct displacements of angulation, translation, rotation, or any combination thereof, within bodily tissues. A fixation apparatus, such as orthopedic fixator 100, utilized with the techniques described herein, can correct a plurality of such displacement defects individually or simultaneously. However, it should be appreciated that the fixation apparatus is not limited to the illustrated orthopedic fixator 100, and that the fixation apparatus can be alternatively constructed as desired. For example, the fixation apparatus can include additional fixation members, can include fixation members having alternative geometries, can include more or fewer adjustment members, can include alternatively constructed adjustment members, or any combination thereof.

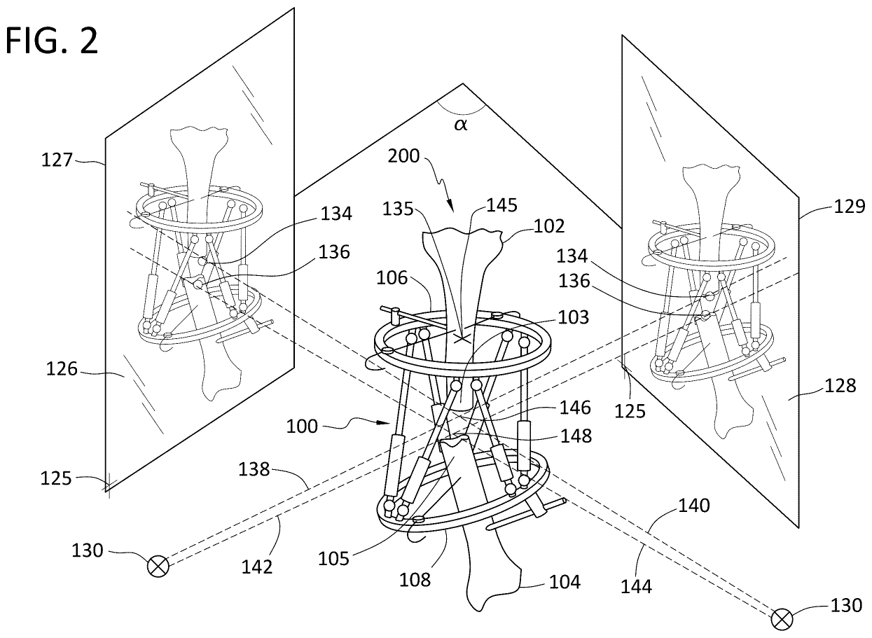

FIG. 2 is a perspective view of an example imaging process of the fixation assembly illustrated in FIG. 1. Referring now to FIG. 2, an example imaging of a fixation apparatus will now be described in detail. The images can be captured using the same or different imaging techniques. For example, the images can be acquired using x-ray imaging, computer tomography, magnetic resonance imaging, ultrasound, infrared imaging, photography, fluoroscopy, visual spectrum imaging, or any combination thereof.

The images can be captured from any position and/or orientation with respect to each other and with respect to the fixator 100 and the anatomical structure segments 102, 104. In other words, there is no requirement that the captured images be orthogonal with respect to each other or aligned with anatomical axes of the patient, thereby providing a surgeon with near complete flexibility in positioning the imagers 130. Preferably, the images 126, 128 are captured from different directions, or orientations, such that the images do not overlap. For example, in the illustrated embodiment, the image planes of the pair of images 126, 128 are not perpendicular with respect to each other. In other words, the angle α between the image planes of the images 126, 128 is not equal to 90 degrees, such that the images 126, 128 are non-orthogonal with respect to each other. Preferably, at least two images are taken, although capturing additional images may increase the accuracy of the method.

The images 126, 128 can be captured using one or more imaging sources, or imagers, for instance the x-ray imagers 130 and/or corresponding image capturing devices 127, 129. The images 126, 128 can be x-ray images captured by a single repositionable x-ray imager 130, or can be captured by separately positioned imagers 130. Preferably, the position of the image capturing devices 127, 129 and/or the imagers 130 with respect to the space origin 135 of the three-dimensional space, described in more detail below, are known. The imagers 130 can be manually positioned and/or oriented under the control of a surgeon, automatically positioned, for instance by a software assisted imager, or any combination thereof. The fixator 100 may also have a respective fixator origin 145.

Techniques used to treat fractures and/or deformities of anatomical structures, such as bones, can include the use of external fixators, such as hexapods and other fixation frames, which are surgically mounted to anatomical structure segments on opposed sides of a fracture site. A pair of radiographic images is taken of the fixator and anatomical structure segments at the fracture site. Data from the images is then manipulated to construct a three-dimensional representation of the fixator and the anatomical structures segments that can be used in developing a treatment plan, which may for example comprise realigning the anatomical structure segments through adjustments to the fixator.

Existing techniques for controlling fixator manipulation may, however, involve a number of limitations that may introduce inefficiency, complication, and unreliability. For example, some conventional techniques may rely on a surgeon or other user to indicate locations of certain fixator elements, such as hinges, within images that are displayed in a graphical user interface of a computer. However, it may often be difficult for the user to identify and mark positions of the hinges and other fixator elements within the images. In particular, depending upon the location and orientation from which an image is captured, hinges and other fixator elements may not be identified easily, such as because they may wholly or partially overlap one another or may otherwise be obscured within the images. This may make it cumbersome for the user to identify the fixator elements, thereby increasing time required to identify the elements, increasing the probability of errors, and reducing the reliability of the calculations. This may reduce the reliability of the treatment plan, possibly resulting in improper alignment of anatomical structures segments during the healing process, compromised union between the anatomical structure segments, necessitating additional rounds of radiographic imaging to facilitate alignment corrections, or even necessitating additional surgical procedures.

U.S. Pat. No. 11,334,997 ('997 patent), issued on May 17, 2022, titled “HINGE DETECTION FOR ORTHOPEDIC FIXATION”, the contents of which is hereby incorporated by reference for all purposes as if fully set forth herein, discloses an automatic method for the detection of hinges and adjustment members using classic image processing algorithms of facilitate the preparation of a treatment plan. The '997 provides additional details regarding the workflow process of taking images of an in place orthopedic fixator, performing frame matching, and the developing a treatment plan based upon the frame matching information and the desired treatment parameters and outcome.

An embodiment of another automatic method for the detection of hinges and fixation members using machine learning, for example neural networks, will be described herein.

Neural networks (NN), including convolutional neural networks (CNN), region based CNN (R-CNN), YOLO (You Only Look Once), and deep learning neural networks may be used to analyze images of the orthopedic fixator (with or without the anatomical structure segments) to determine the location of the hinges and adjustment members. A neural network would be trained to generate a proposal for frame matching. This training requires a large number of training examples including images that have associated frame matching information. The data set used to train the neural network may be divided into training, validation and test sets. For example, certain images may be used for training but not for validation or testing, while other images are used for validation only or testing only. During training of the neural network, the training data is used iteratively to compute the gradient and update the network weights and biases. The validation set is used to calculate a validation error, and training may continue until a minimum validation error is achieved. Then the test data may then be used to determine a test error. This test error may be used to compare different models or determine hyperparameters, including learning rate, optimizer, cost function, etc.

The number of labelled images available, i.e., images of orthopedic fixators with frame matching data, may not be sufficient to adequately train the neural network. The number of labelled images may further be limited by privacy issues. As a result, it is proposed that simulated X-rays of orthopedic fixators be generated to provide sufficient labelled training data to the machine learning model. Artificial X-Rays allow for the creation of a large number of images with modification of various parameters (e.g., frame configuration, X-ray setup and intensity, image resolution, noise, aspect ratio, image annotations (text), contrast and brightness, center of the frame, blur, etc.). The content can be automatically labeled because the configuration of the frame is known and various features in the artificial X-ray can be identified and labelled.

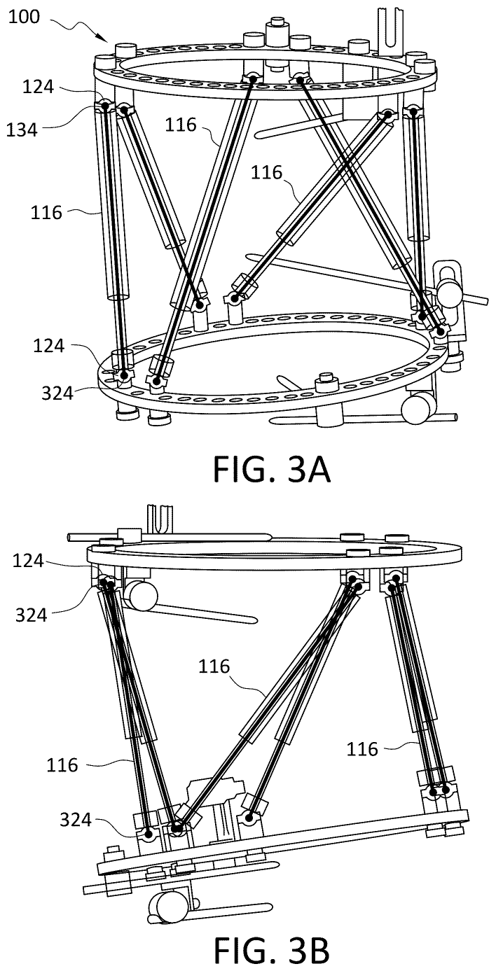

FIGS. 3A and 3B illustrate generally anterior and lateral view X-rays with user annotations on the adjustable length struts 116. Although only a few examples are shown, the invention is not limited as such, and it is contemplated that the invention will ingest series of sequential or overlapping S-ray images that are combined or “stacked” (“stacked frames.”).

In the orthopedic fixture 100 there are six adjustable length struts 116 present. Each of these adjustable length struts 116 have been annotated by a user. Specially the universal joints (or hinges) 124 have been annotated using dots 324. The location of these dots 324 will allow for the planning software to determine a treatment plan based upon the configuration of the orthopedic fixture 100. As described above, annotating these images can be time consuming and may be lacking in accuracy.

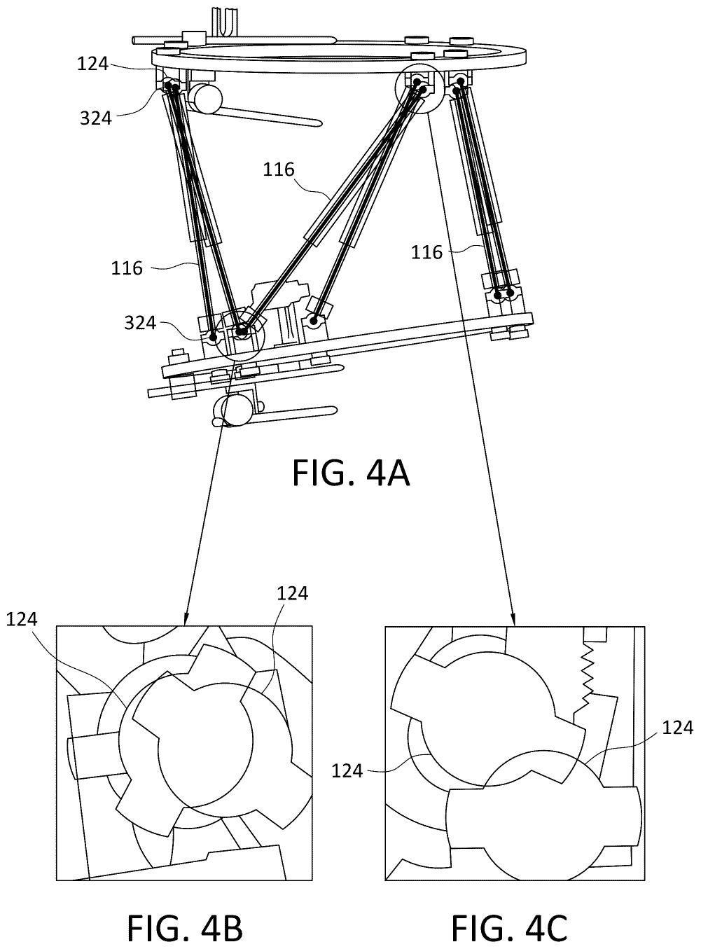

FIG. 4A illustrates the same lateral view as FIG. 3B with overlapping universal joints highlighted. FIGS. 4B and 4C illustrate the specific regions of the image in FIG. 4A that illustrate where universal joints 124 overlap and are difficult to identify and annotate. The use of classical image processing as in the '997 patent may struggle to differentiate such overlapping universal joints. Hence there is a need for a different approach such as the use of machine learning models such as neural networks. Such a model can be trained to understand such overlapping images and still provide an accurate frame matching for use in generating a treatment plan.

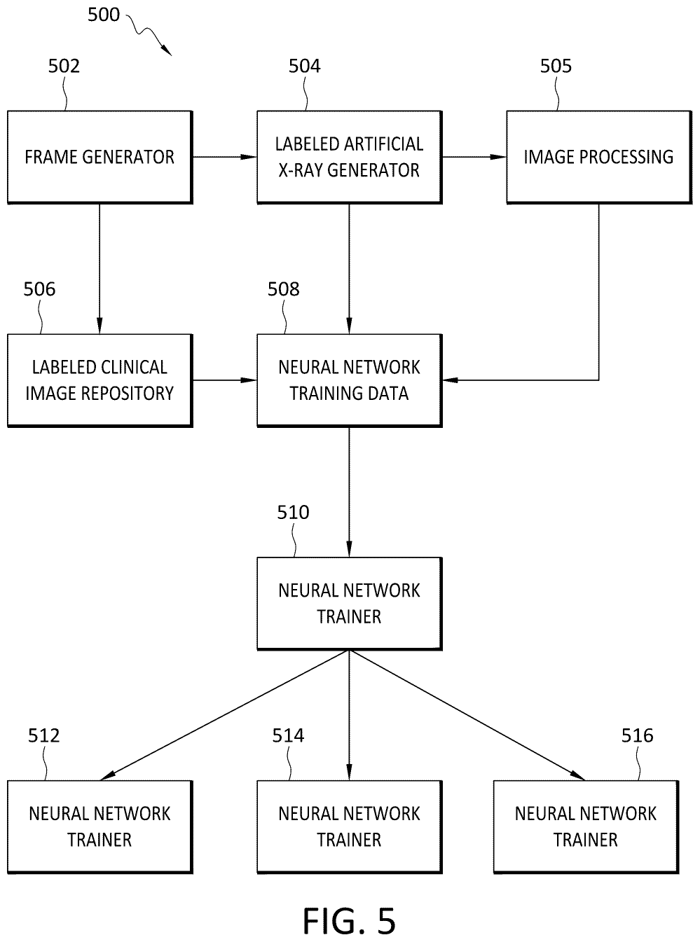

FIG. 5 illustrates an automatic feature matching system and method using neural networks. The automatic feature matching system 500 includes a frame generator 502, labelled artificial X-ray generator 504, labelled clinical images repository 506, neural network training data 508, neural network trainer 510, full frame detection neural network 512, hinge and adjustment member detection neural network 514, and two image detection neural network 516.

The frame generator 502 generates example frames. This may be done by randomly generating fixation frames that make clinical sense. These may be based upon statistics drawn from clinical data describing frames in use. For example, the number length adjustable struts 116 may be selected randomly within a specific range of numbers. Further, this random selection may be based upon a probability distribution of the number of length adjustable struts 116 found in the clinical data. The types and orientation of the fixator ring 108 may also be randomly selected within a specified range of values. Then the length adjustable struts 116 may be randomly connected between the fixator ring 108 in a clinically reasonable manner. The frame generator 502 may generate the number of frames needed to generate sufficient training data to properly train the neural network. In some embodiments, anatomical structure segments may be added to the frames. This may also be done randomly based upon clinical data. Generated frames may include randomly selected components, such as screws, bolts, fixator elements, wires, posts, etc. The generated frames are preferably as realistic as possible and should provide robust learning of components.

The artificial X-ray generator 504 receives the generated frames from the frame generator 502 and produces artificial X-rays, which may be labelled, of the generated frames. This may be done from various angles with different X-ray setups using different parameters, e.g., image resolutions, noise, etc. The number of artificial X-rays generated should provide a sufficient number of training data points to train the neural network. Labelling information may include key words, coordinates, and other information used to identify regions of interest, and may be created and stored in parallel. Labelling information may be stored separate from the generated artificial X-ray image data, such as, for example, in a separate database, text file, or other means. The operation of the artificial X-ray generator 504 will be described in greater detail below.

According to embodiments of the invention, images may be further manipulated for training purposes. Image processing 505 can be performed to add blurring, alter aspect ratio, introduce noise, add annotations, etc., to s mimic real-world conditions, enhance training datasets for machine learning, or test image-processing algorithms. The following is a non-limiting listing of further image processing that may be performed:
Blurring (Gaussian, median, and motion blur) to simulate out-of-focus images or motion blur.
Aspect Ratio changes to the image dimensions to simulate different capture resolutions or imaging perspectives.
Add Random noise (Gaussian, salt-and-pepper, etc.) to mimic imaging sensor noise or environmental factors.
Add Annotations (e.g., labels, markers, overlays, bounding boxes or segmentation masks) to areas of interest.
Adjusting brightness, contrast, or gamma levels to simulate variations in lighting conditions or image quality.
Alter rotation, scaling, and translation (image orientation, zoom level, or object location).
Alter color/grayscale to simulate different imaging modes.
Image Cropping to focus on specific areas or simulate framing variations.
   The raw labelled X-rays and/or the processed labelled X-rays can be used as training data.

The labelled clinical images repository 506 includes clinical images that have been labelled for frame matching purposes. This data will typically be anonymized to protect the privacy of the patients. The labelled data may include different types of frame matching data, such as the location of the hinges, location of the adjusting struts, location of the rings, etc. or any combination thereof. It is beneficial that this labelled data includes accurate annotations of the frame matching elements in order to best train the neural network.

For the artificially generated X-ray images, labeling information may be stored and provided in an additional image and dataset, where content of each pixel (e.g. hinge+hinge no. & distal ring) is encoded depending on what frame feature contributed to the pixel's content. Labelling data may be stored separately in a database, text file, etc. From there a second subset of data for training may be created, e.g., the labeling image will be used to identify hinges, create regions of interest (ROIs) around these hinges and use these for training on hinge only or struts may be identified and ROIs be created around the struts and this is used for training. By having this detailed encoded image (or data matrix’) data may be selected for specific training of the different approaches.

The neural network training data 508 is the data that is used to train, validate, and test the neural network. The training data may include any mix of labelled clinical images or artificially generated, labelled X-rays (raw and/or image processed) depending on the number of labelled clinical images available. It is possible that the frame matching accuracy of the artificially generated X-rays is higher than that of the labelled clinical images because of the limitation of human annotation of the images. In one embodiment, artificially generated, labelled X-rays (raw and/or image processed) X-rays may be used for training the neural network with the labelled clinical images being used to validate the training of the model and to test the model. In another embodiment, a mix of labelled clinical images and artificially generated X-rays may be used for training, validation, and training. Other mixes may be contemplated as well. In another embodiment, Transfer Learning may be employed. For example, the network may be trained on artificial X-Rays, and then the trained model be used to train another model with the clinical images.

The neural network trainer 510 then trains a neural network using the neural network training data 508. This may be done using various known methods. It may include multiple training runs with different hyperparameters. Further, different model architectures may be selected and trained and the architecture with the best performance selected. The neural network trainer 510 may train and produce three different types of models. The first may be a hinge and adjustment member detection neural network 514. This model determines the locations of the just the hinges or adjustment members of the frame. The locations of the hinges or adjustment members may then be used to match the frame to the image and anatomy so that the software tools can then generate a treatment plan. This may be done for example as described in the '997 patent. The hinge and adjustment member detection neural network 514 may still struggle to separate hinges in some situations because of overlap. In such a situation, the second model, the full frame detection neural network 512, may be used.

The full frame detection neural network 512 trains a neural network based upon the complete frame data annotated in either the labelled clinical images or the artificially generated X-rays. For the full frame detection neural network 512 the artificially generated X-rays may prove especially useful as such data is readily generated from the frame generator. By using the rings, adjustable struts in addition to the hinges a better frame matching may be possible when the hinges overlap in the available images.

The third neural network analyzes a first image of an image pair (e.g., anterior and lateral) to then analyzes a second image of the pair based on the analysis of the first image. For example, if one of the anterior images is easier to analyze, then that image is first analyzed and then the second image is analyzed based upon the analysis of the first image. Such a situation may provide improved matching results. In some situations when the full frame detection neural network 512 does not provide adequate results the two image detection neural network 516 may be used.

In various embodiments, one or some combination of the full frame detection neural network 512, hinge and adjustment member detection neural network 514, and two image detection neural network 516 may be used to analyze images. For example, the models may be used in the progression described above, but they may be used in other orders and combinations as well.

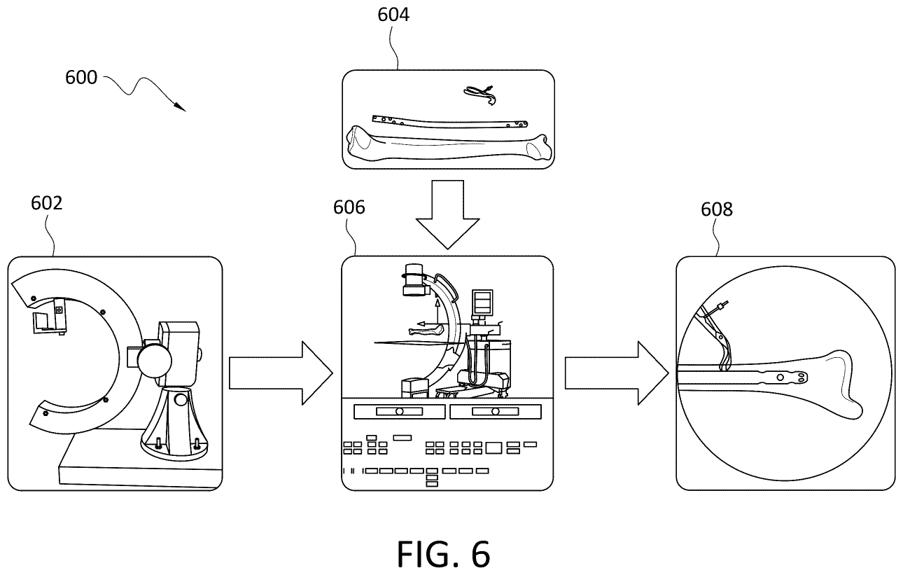

FIG. 6 illustrates a block diagram of one example artificial X-ray generator according to embodiments of the present invention. The artificial X-ray generator 600 includes kinematic model 602, subject 3D models 604, and image generator 606. Here, models refer to simulations of physical objects and their behavior, not to deep learning models. The kinematic model 602 describes the motion that the X-ray machine can carry out. Such a kinematic model 602 may model different brands, models, and types of X-ray machines. The subject 3D models 604 include 3D models of various anatomy including bones and other tissue, and medical devices and implants, such as plates, screws, joint implants, fixture frames, etc. The subject 3D models 604 may receive input from the frame generator 502 of the automatic feature matching system 500. Included with the 3D model is information regarding the material makeup of the subjects. The image generator 606 takes the kinematic model 602 and information from subject 3D models 604 to generate the artificial X-ray 608.

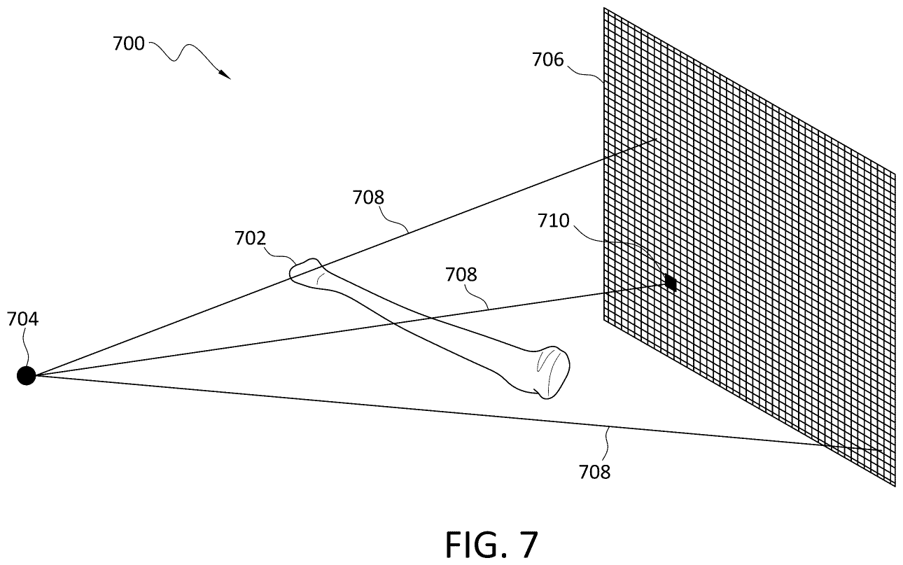

FIG. 7 illustrates an artificial X-ray system 700 according to embodiments of the present invention. The artificial X-ray system 700 includes an artificial X-ray source 704 impinging on an artificial X-ray detector 706 through a bone 702. X-ray paths 708 are traced from the artificial X-ray source 704 to each pixel of the artificial X-ray detector 706. The artificial X-ray system 700 spatially traces along each X-ray path 708 to determine if the X-ray path 708 passes through any object. If so then information regarding the object, in this case the bone 702, is used to calculate intensity value for a X-ray detector pixel 710. This is done for each of the pixels in the artificial X-ray detector 706 to generate an artificial X-ray.

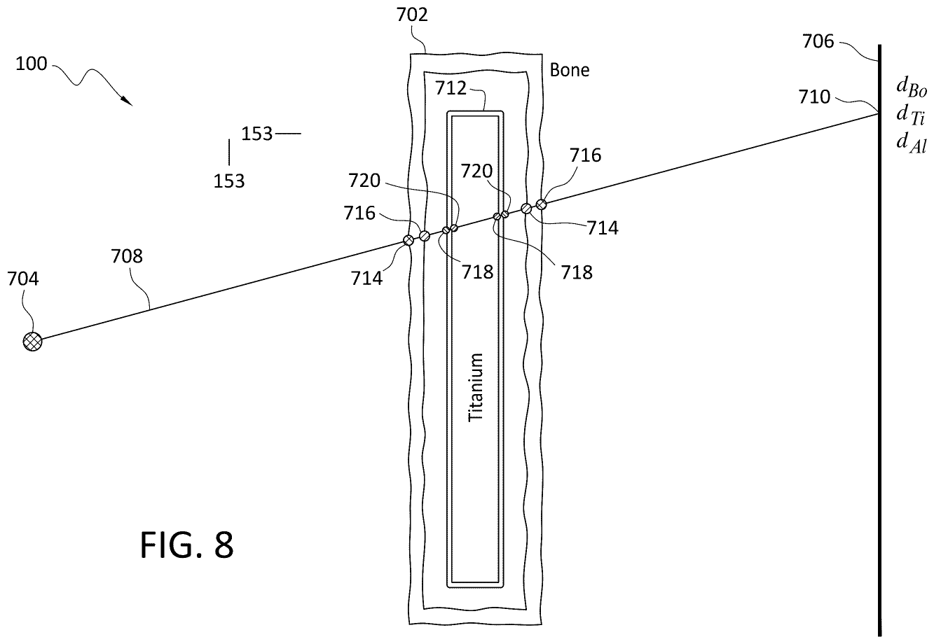

FIG. 8 illustrates a X-ray path 708 passing through different objects on its way to the artificial X-ray detector 706. In this case the object is a bone 702 with an implant 712 inside the bone. So the artificial X-ray system 700 determines when the X-ray path 708 passes through any objects. In this case, the X-ray path 708 enters the bone 702 at bone entry point 714 and exits at 716. Then it enters the implant 712 at implant entry point 718 and exits at implant exit point 720. Then the X-ray path 708 again enters the implant 712 at implant entry point 718 and exits at implant exit point 720. The X-ray path 708 next enters the bone 702 at bone entry point 714 and exits at bone exit point 716. The X-ray path 708 then extends to the X-ray detector pixel 710 of the artificial X-ray detector 706. The artificial X-ray system 700 can then determine the path length along the X-ray path 708 for each object based upon the locations where the X-ray path 708 enters and exits the object. In FIG. 8 the distances between the two instances of bone entry point 714 and bone exit point 716 may be determined and summed to determine the path length of X-ray path 708 through the bone 702. The same can be done for the implant 712. Then using these lengths and knowing the material of the object, an attenuation value for each of the object may be determined and used to calculate the intensity of the virtual X-ray beam along X-ray path 708 at X-ray detector pixel 710. For example, the Beer-Lambert Law could be applied to quantify how much X-ray radiation is absorbed or transmitted by different tissues in the body, such as bone, muscle, or fat, to generate intensity. Different X-ray intensities may be used for the artificial X-ray source 704 and different sensitivities and image resolutions may be used for the artificial X-ray detector 706 in order to model different X-ray systems.

The artificial X-ray generator 504 may use the artificial X-ray system 700 to generate artificial X-rays of various randomly generated fixation systems. As discussed above, various example orthopedic fixators may be generated and then an artificial X-ray may be taken of that system. The artificial X-rays may be generated from different aspects of the orthopedic fixator as well as with different X-ray system characteristics.

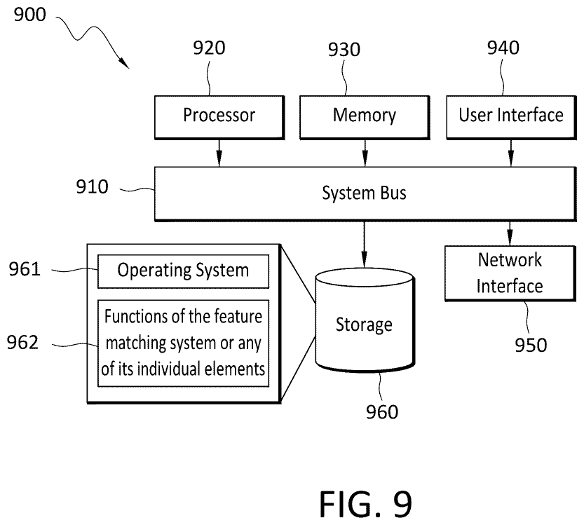

FIG. 9 illustrates an exemplary hardware diagram 900 for implementing automatic feature matching system 500, frame generator 502, artificial X-ray generator 504, neural network trainer 510, full frame detection neural network 512, adjustment member detection neural network 514, image detection neural network 516, or artificial X-ray system 700. As shown, the device 900 includes a processor 920, memory 930, user interface 940, network interface 950, and storage 960 interconnected via one or more system buses 910. It will be understood that FIG. 9 constitutes, in some respects, an abstraction and that the actual organization of the components of the device 900 may be more complex than illustrated.

The processor 920 may be any hardware device capable of executing instructions stored in memory 930 or storage 960 or otherwise processing data. As such, the processor may include a microprocessor, microcontroller, graphics processing unit (GPU), neural network processor, field programmable gate array (FPGA), application-specific integrated circuit (ASIC), or other similar devices. The processor may be a secure processor or include a secure processing portion or core that resists tampering.

The memory 930 may include various memories such as, for example L1, L2, or L3 cache or system memory. As such, the memory 930 may include static random-access memory (SRAM), dynamic RAM (DRAM), flash memory, read only memory (ROM), or other similar memory devices. Further, some portion or all of the memory may be secure memory with limited authorized access and that is tamper resistant.

The user interface 940 may include one or more devices for enabling communication with a user such as a surgeon or other medical professional. For example, the user interface 940 may include a display, a touch interface, a mouse, and/or a keyboard for receiving user commands. In some embodiments, the user interface 940 may include a command line interface or graphical user interface that may be presented to a remote terminal via the network interface 950.

The network interface 950 may include one or more devices for enabling communication with other hardware devices. For example, the network interface 950 may include a network interface card (NIC) configured to communicate according to the Ethernet protocol or other communications protocols, including wireless protocols. Additionally, the network interface 950 may implement a TCP/IP stack for communication according to the TCP/IP protocols. Various alternative or additional hardware or configurations for the network interface 950 will be apparent.

The storage 960 may include one or more machine-readable storage media such as read-only memory (ROM), random-access memory (RAM), magnetic disk storage media, optical storage media, flash-memory devices, or similar storage media. In various embodiments, the storage 960 may store instructions for execution by the processor 920 or data upon with the processor 920 may operate. For example, the storage 960 may store a base operating system 961 for controlling various basic operations of the hardware 900. The storage 962 includes instructions for implementing the functions of the automatic feature matching system 500 or any of its elements in any combination thereof.

It will be apparent that various information described as stored in the storage 960 may be additionally or alternatively stored in the memory 930. In this respect, the memory 930 may also be considered to constitute a “storage device” and the storage 960 may be considered a “memory.” Various other arrangements will be apparent. Further, the memory 930 and storage 960 may both be considered to be “non-transitory machine-readable media.” As used herein, the term “non-transitory” will be understood to exclude transitory signals but to include all forms of storage, including both volatile and non-volatile memories.

The system bus 910 allows communication between the processor 920, memory 930, user interface 940, storage 960, and network interface 950.

While the host device 900 is shown as including one of each described component, the various components may be duplicated in various embodiments. For example, the processor 920 may include multiple microprocessors that are configured to independently execute the methods described herein or are configured to perform steps or subroutines of the methods described herein such that the multiple processors cooperate to achieve the functionality described herein. Further, where the device 900 is implemented in a cloud computing system, the various hardware components may belong to separate physical systems. For example, the processor 920 may include a first processor in a first server and a second processor in a second server.

The foregoing disclosure provides illustration and description but is not intended to be exhaustive or to limit the aspects to the precise form disclosed. Modifications and variations may be made in light of the above disclosure or may be acquired from practice of the aspects.

As used herein, the term “component” is intended to be broadly construed as hardware, firmware, and/or a combination of hardware and software. As used herein, a processor is implemented in hardware, firmware, and/or a combination of hardware and software.

As used herein, satisfying a threshold may, depending on the context, refer to a value being greater than the threshold, greater than or equal to the threshold, less than the threshold, less than or equal to the threshold, equal to the threshold, not equal to the threshold, and/or the like. It will be apparent that systems and/or methods described herein may be implemented in different forms of hardware, firmware, and/or a combination of hardware and software. The actual specialized control hardware or software code used to implement these systems and/or methods is not limiting of the aspects. Thus, the operation and behavior of the systems and/or methods were described herein without reference to specific software code-it being understood that software and hardware can be designed to implement the systems and/or methods based, at least in part, on the description herein.

As used herein, the term “non-transitory machine-readable storage medium” will be understood to exclude a transitory propagation signal but to include all forms of volatile and non-volatile memory. When software is implemented on a processor, the combination of software and processor becomes a specific dedicated machine.

Because the data processing implementing the embodiments described herein is, for the most part, composed of electronic components and circuits known to those skilled in the art, circuit details will not be explained in any greater extent than that considered necessary as illustrated above, for the understanding and appreciation of the underlying concepts of the aspects described herein and in order not to obfuscate or distract from the teachings of the aspects described herein.

Unless stated otherwise, terms such as “first” and “second” are used to arbitrarily distinguish between the elements such terms describe. Thus, these terms are not necessarily intended to indicate temporal or other prioritization of such elements.

It should be appreciated by those skilled in the art that any block diagrams herein represent conceptual views of illustrative hardware embodying the principles of the aspects.

While each of the embodiments are described above in terms of their structural arrangements, it should be appreciated that the aspects also cover the associated methods of using the embodiments described above.

Unless otherwise indicated, all numbers expressing parameter values and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by embodiments of the present disclosure. As used herein, “about” may be understood by persons of ordinary skill in the art and can vary to some extent depending upon the context in which it is used. If there are uses of the term which are not clear to persons of ordinary skill in the art, given the context in which it is used, “about” may mean up to plus or minus 10% of the particular term.

Even though particular combinations of features are recited in the claims and/or disclosed in the specification, these combinations are not intended to limit the disclosure of various aspects. In fact, many of these features may be combined in ways not specifically recited in the claims and/or disclosed in the specification. Although each dependent claim listed below may directly depend on only one claim, the disclosure of various aspects includes each dependent claim in combination with every other claim in the claim set. A phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

No element, act, or instruction used herein should be construed as critical or essential unless explicitly described as such. Also, as used herein, the articles “a” and “an” are intended to include one or more items and may be used interchangeably with “one or more.” Furthermore, as used herein, the terms “set” and “group” are intended to include one or more items (e.g., related items, unrelated items, a combination of related and unrelated items, and/or the like), and may be used interchangeably with “one or more.” Where only one item is intended, the phrase “only one” or similar language is used. Also, as used herein, the terms “has,” “have,” “having,” and/or the like are intended to be open-ended terms. Further, the phrase “based on” is intended to mean “based, at least in part, on” unless explicitly stated otherwise.

1. An automatic feature matching system for orthopedic fixators, comprising:
a frame generator configured to generate a simulation of an orthopedic fixator system;
an artificial X-ray generator configured to generate a plurality of artificial X-ray images including the generated simulation of the orthopedic fixator system in the plurality of artificial X-ray image, wherein the plurality of artificial X-ray images are labelled;
a neural network training data including the plurality of labelled artificial X-ray images; and
a neural network trainer configured to be trained based on the plurality of labelled artificial X-ray images;
wherein the neural network trainer is configured to generate a frame detection neural network that is configured to detect orthopedic fixator system features in real X-ray images input into the frame detection neural network.

2. The system of claim 1, wherein the frame generator randomly selects orthopedic fixator parameters and configurations based upon clinical data.

3. The system of claim 1, wherein the frame generator further generates anatomy elements connected to the generated orthopedic fixator.

4. The system of claim 1, wherein the artificial X-ray generator generates multiple X-rays of the generated orthopedic frame using different aspects and X-ray parameters.

5. The system of claim 1, further comprising labelled clinical images and datasets of orthopedic fixators.

6. The system of claim 5, wherein the neural network training data includes the labelled clinical images of orthopedic fixators.

7. The system of claim 6, wherein at least a subset of the labelled clinical images of orthopedic fixators are used by the neural network trainer to validate the frame detection neural network.

8. The system of claim 6, wherein at least a subset of the labelled clinical images of orthopedic fixators are used by the neural network trainer to test the frame detection neural network.

9. The system of claim 6, wherein at least a subset of the labelled clinical images of orthopedic fixators are used by the neural network trainer to validate the frame detection neural network.

10. The system of claim 1, wherein the generated frame detection neural network is configured to detect hinges and their positions in the images input into the frame detection neural network.

11. The system of claim 1, wherein the generated frame detection neural network is configured to adjustment members in the images input into the frame detection neural network.

12. The system of claim 1, wherein the generated frame detection neural network is configured to detect hinges and adjustment members in the images input into the frame detection neural network.

13. The system of claim 1, wherein the generated frame detection neural network is configured to detect a full frame of the orthopedic fixator in the images input into the frame detection neural network.

14. The system of claim 1, wherein the generated frame detection neural network is configured to detect features in two images input into the frame detection neural network.

15. The system of claim 1, further comprising:
an image processor configured to apply image processing to one or more of said plurality of labelled artificial X-ray images to generate a plurality of processed labelled artificial X-ray images;
wherein the neural network training data further includes the plurality of processed labelled artificial X-ray images, and
the neural network trainer is configured to be trained based on the plurality of labelled artificial X-ray images and/or the plurality of processed labelled artificial X-ray images.

16. The system of claim 15, wherein the image processor is configured to apply image processing including at least one of blurring, aspect ratio, noise, annotations, brightness, contrast, rotation, scaling, translation, color, and cropping.

17. The system of claim 1, wherein the neural network trainer generates one or more trained models configured to detect orthopedic fixators in an image.

18. The system of claim 17, wherein the trained model is used to train a new model for detecting orthopedic fixators using a new neural network training dataset.

19. A method of generating a deep learning model for automatic feature matching of orthopedic fixators, comprising steps of:
generating a simulation of an orthopedic fixator system;
generating a plurality of artificial X-ray images including the generated simulation of the orthopedic fixator system in the plurality of artificial X-ray image, wherein the plurality of artificial X-ray images are labelled;
creating neural network training data including the plurality of labelled artificial X-ray images; and
training a neural network model based on the plurality of labelled artificial X-ray images to generate a frame detection neural model that is configured to detect orthopedic fixator system features in real X-ray images input into the frame detection neural network.

20. The method of claim 19, wherein the generating a simulation step randomly selects orthopedic fixator parameters and configurations based upon clinical data.

21. The method of claim 19, wherein the generating a simulation step further generates anatomy elements connected to the generated orthopedic fixator.

22. The method of claim 19, wherein the generating a plurality of artificial X-ray images step generates multiple X-rays of the generated orthopedic frame using different aspects and X-ray parameters.

23. The method of claim 19, further comprising labelled clinical images and datasets of orthopedic fixators.

24. The method of claim 19, wherein the neural network training data includes the labelled clinical images of orthopedic fixators.

25. The method of claim 19, wherein at least a subset of the labelled clinical images of orthopedic fixators are used by the neural network trainer to validate the frame detection neural network.

26. The method of claim 19, wherein at least a subset of the labelled clinical images of orthopedic fixators are used by the neural network trainer to test the frame detection neural network.

27. The method of claim 26, wherein at least a subset of the labelled clinical images of orthopedic fixators are used by the neural network trainer to validate the frame detection neural network.

28. The method of claim 19, wherein the generated frame detection neural model is configured to detect hinges and their positions in the images input into the frame detection neural network.

29. The method of claim 19, wherein the generated frame detection neural model is configured to adjustment members in the images input into the frame detection neural network.

30. The method of claim 19, wherein the generated frame detection neural model is configured to detect hinges and adjustment members in the images input into the frame detection neural network.

31. The method of claim 19, wherein the generated frame detection neural model is configured to detect a full frame of the orthopedic fixator in the images input into the frame detection neural network.

32. The method of claim 19, wherein the generated frame detection neural model is configured to detect features in two images input into the frame detection neural network.

33. The method of claim 19, further comprising:
image processing one or more of said plurality of labelled artificial X-ray images to generate a plurality of processed labelled artificial X-ray images;
wherein the neural network training data further includes the plurality of processed labelled artificial X-ray images, and the model is based on the plurality of labelled artificial X-ray images and/or the plurality of processed labelled artificial X-ray images.

34. The method of claim 33, wherein image processing includes at least one of blurring, aspect ratio, noise, annotations, brightness, contrast, rotation, scaling, translation, color, and cropping.

35. The method of claim 19, wherein the method generates one or more trained models configured to detect orthopedic fixators in an image.

36. The method of claim 19, wherein the trained model is used to train a new model for detecting orthopedic fixators using a new neural network training dataset.
